(19) United States
(12) Patent Application Publication
(10) Pub. No.: US 2020/0323693 A1
(43) Pub. Date: Oct. 15, 2020

(54) INTERVENTIONAL DEVICE DRESSING SYSTEM

(71) Applicant: pfm Medical, Inc., Carlsbad, CA (US)

(72) Inventor: Jiaye Jho, Carlsbad, CA (US)

(21) Appl. No.: 16/913,182

(22) Filed: Jun. 26, 2020

Related U.S. Application Data
(63) Continuation-in-part of application No. 16/149,537, filed on Oct. 2, 2018.

(51) Int. Cl. A61F 13/02; A61F 13/00; A61L 15/22; A61L 15/58

(52) U.S. Cl. CPC A61F 13/02; A61F 13/00063; A61L 15/22; A61L 15/58; A61L 2300/206; A61F 2013/00472; A61F 2013/00646; A61L 2300/404; A61F 2013/00412

(57) ABSTRACT

A wound or access location dressing device can be used with a transcutaneous medical device such as a cannula, a catheter, or other transcutaneous interventional device. The dressing device is formed at least partially of a flexible hydrophillic polyurethane matrix, an antimicrobial agent contained within or coupled to the matrix, and a hemostatic agent contained within or coupled to the matrix.

### CROSS REFERENCE TO RELATED APPLICATION

This application is continuation-in-part of U.S. patent application Ser. No. 16/149,537, filed Oct. 2, 2018. Priority of the aforementioned filing date is claimed and the patent application is incorporated herein by reference in its entirety.

### BACKGROUND

Disclosed is a wound dressing system that can be used with intravenous (IV) catheter systems and other percutaneous devices.

Vascular and nonvascular percutaneous medical devices such as IV catheters and drainage tubes are widely used with patients. The catheters or other percutaneous devices are inserted into a patient via an access location through the skin. This can provide an entryway for disease and is also a location of possible hemorrhage and/or thrombosis. As a result, there is typically a need to provide a dressing around the access location for antimicrobial purposes and to deal with bleeding.

There remains a need for an effective dressing for use with IV catheters that stops bleeding and is an effective antimicrobial solution.

### SUMMARY

The disclosed system is a wound or access location dressing device for use with a transcutaneous medical device such as a cannula, a catheter, or other transcutaneous interventional device. In an example embodiment, the dressing device is formed at least partially of a flexible hydrophillic polyurethane matrix, an antimicrobial agent contained within or coupled to the matrix, and a hemostatic agent contained within or coupled to the matrix. The dressing advantageously prevents microbial colonization of the dressing and stops or inhibits bleeding from the access location site without adversely affecting wound healing.

The antimicrobial agent can be, for example, chlorhexidine gluconate (CHG)(or a salt thereof) such as in an amount to achieve an antimicrobial effect without adversely affecting wound healing.

The hemostatic agent may be, for example, kaolin, an inorganic mineral that accelerates the body's natural clotting ability and produces no exothermic reaction thereof in an amount to achieve a hemostatic effect.

The wound dressing can contain a broad-spectrum antimicrobial agent and a hemostatic agent with a moisture vapour permeable backing. The dressing can include one or more holes, slits, or cut-outs to allow insertion of an IV catheter line or other percutaneous or interventional device therethrough. The dressing contains sufficient quantities of the antimicrobial agent to ensure that a clear antimicrobial zone of inhibition can be maintained around the insertion site and to prevent microbial contamination of the dressing. The device also contains sufficient quantities of hemostatic agent in order to successfully control minor bleeding at the insertion site.

Other features and advantages should be apparent from the following description of various embodiments, which illustrate, by way of example, the principles of the disclosure.

### BRIEF DESCRIPTION OF THE DRAWINGS

FIG. 1 shows a schematic representation of a wound dressing device.

### DETAILED DESCRIPTION

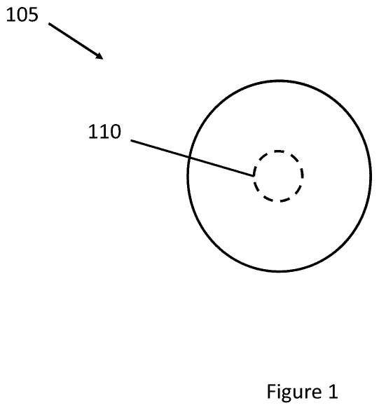

FIG. 1 shows a schematic representation of a wound dressing 105 that is configured to control or regulate bleeding at the access sites of an interventional device, such as an IV catheter, percutaneous device, or other interventional device that extends through skin of a patient at the access site. The access site may be formed by a cut-down, puncture, skin opening, etc. The dressing contains, is coated with, or is otherwise coupled to an antimicrobial agent to help resist microbial colonization of the dressing. The dressing also contains, is coated with, or is otherwise coupled to a hemostatic agent such that it is configured to reduce a time of bleeding at the access device. The dressing may also include a moisture vapour permeable backing to allow for moisture transmission. The backing may, for example, comprise a thin polyurethane film.

The antimicrobial agent can be, for example, chlorhexidine gluconate (CHG), such as in an amount to achieve an antimicrobial effect without adversely affecting wound healing. The hemostatic agent may be, for example, kaolin, an inorganic mineral that accelerates the body's natural clotting ability and produces no exothermic reaction thereof in an amount to achieve a hemostatic effect.

The dressing may be secured to the access site in any of a variety of manners. In an embodiment, the dressing includes an adhesive component. The dressing may also include a component that renders the dressing generally sterile. Sterilization may be carried out, for example, using gamma irradiation but other sterilization methods such as ethylene oxide sterilization may also be used.

With reference still to FIG. 1, the wound dressing 105 may be a planar or bulk body that is sized and shaped to be positioned at, near, or around an access site of a patient. The dressing 105 may have at least one skin contacting surface that at least partially contacts a patient's skin at or near the location of the access site when in use.

The dressing 105 may include one or more holes, openings, slits, or cutouts 110 through which at least a portion of the interventional device can be positioned when the dressing is in place at the access site. Such holes, openings, slits, or cutouts 110 may be contained entirely within an outer perimeter of the dressing 105 as shown or may intersect the outer perimeter. The dressing may have any of a variety of shapes. In an embodiment, the holes, openings, slits, or cutout is sized and shaped to seal around an outer contour shape of the interventional device, such as a catheter, which is considered as part of this disclosure. The interventional device may have a circular, non-circular, or irregular cross-sectional shape. The holes, openings, slits, or cutouts can be shaped to conforms or correspond to the outer-cross-sectional shape of the interventional device. The correspondence can be exact in an embodiment to encourage a seal between the opening and the outer contour of the interventional device. In an example, a separate seal member is positioned entirely or partially on or around the contour holes, openings, slits, or cutouts such that the seal member establishes a seal with the outer contour of the interventional device. In another embodiment, there is a gap between an outer edge of the hole, opening, slit, or cutout and the seal member, with the seal member configured to expand and engage in a sealing manner with the interventional device. The seal member can be configured to expand and seal in response to the seal member coming into contact with fluid, such as blood and/or into contact with a microbe. The seal member can be made of an entirely different material than one or more materials of the remainder of the dressing.

In one embodiment, the dressing device has an adhesive on the skin contacting surface to aid in site securement and also for removal and re-securement.

The dressing 105 can effectively maintain antimicrobial efficacy over a period of time. In an example, the dressing can effectively maintain antimicrobial efficacy up to 7 days.

The dressing can be at least partially formed of a polymeric base material, such as polyurethane foam. In a non-limiting example, the dressing can be formed in concentrations of, for example, 8% (w/w) Kaolin, 11% A (w/w) CHG, and 81% hydrophillic flexible polyurethane foam. In another example embodiment, the dressing is formed in a concentration of 5% (w/w) to 15% (w/w) Kaolin, 3% (w/w) to 12% (w/w) Kaolin, or 5% (w/w) to 20% (w/w) Kaolin. In an embodiment, the dressing is formed in a concentration of 15% (w/w) to 25% (w/w) CHG, 10% (w/w) to 25% (w/w) CHG, 13% (w/w) to 28% (w/w) CHG, or 12% (w/w) to 20% (w/w) CHG. A remainder of the concentration can be a foam such as hydrophillic flexible polyurethane foam.

In an example embodiment the wound dressing is circular with an outer diameter of 0.6 to 2 inches.

In an example method of use, an access site is formed through the skin of the patient. As mentioned, the access site may be a surgical cut-down through the skin, a percutaneous puncture, or any other opening through the skin. An interventional device, such as a catheter formed of an elongated body with an internal lumen is then inserted through the access site and into the patient. The dressing is positioned at or near the access site such that the skin contacting surface contacts the skin and at least a portion of the dressing is in contact with the opening in the skin. As mentioned, the dressing maintains antimicrobial efficacy as well as hemostasis at the access location.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A dressing system for use with a transcutaneous medical device, comprising:
a flexible hydrophillic polyurethane matrix;
an antimicrobial agent contained within the matrix, wherein the antimicrobial agent is chlorhexidine gluconate; and
a haemostatic agent contained within the matrix, wherein the haemostatic agent is Kaolin in an amount to achieve a hemostatic effect
wherein the dressing is formed in concentrations of 5% to 15% (w/w) Kaolin, 15%- to 25% (w/w) chlorhexidine gluconate, and a remainder hydrophillic flexible polyurethane foam.

2. The dressing system of claim 1, wherein the dressing has an adhesive on a first surface.

3. The dressing system of claim 1, wherein the dressing has an aperture or slit sized to receive a medical device therethrough.

4. The dressing system of claim 1, wherein the medical device is a catheter.

5. The dressing system of claim 1, wherein the dressing is circular.

6. The dressing system of claim 1, further comprising a transcutaneous medical device.

7. The dressing system of claim 6, wherein the dressing includes an opening.

8. The dressing system of claim 7, wherein the opening of the dressing has a contour that conforms exactly to an outer shape of the interventional device.

9. The dressing system of claim 8, further comprising a seal member positioned on the opening.

10. The dressing system of claim 9, wherein the seal member is made of a material different than a material of the dressing.
